(19) United States
(12) Patent Application Publication
(10) Pub. No.: US 2003/0215475 A1
(43) Pub. Date: Nov. 20, 2003

(54) Dry feeling low spreading emollient gel

(76) Inventors: Anil N. Shah, East Windsor, NJ (US); Timothy C. Morris, Morton, PA (US)

Correspondence Address: COGNIS CORPORATION, 2500 RENAISSANCE BLVD., SUITE 200, GULPH MILLS, PA, 19406

(21) Appl. No.: 10/388,943

(22) Filed: Mar. 14, 2003

Related U.S. Application Data
(60) Provisional application No. 60/365,285, filed on Mar. 19, 2002.

(51) Int. Cl. A61K 7/00; A61K 31/716

(52) U.S. Cl. 424/401; 514/60

(57) ABSTRACT

A dry feeling, low-spreading emollient gel composition containing: (a) an emollient component; (b) a carbohydrate derivative; and (c) a solid particulate component.

### CROSS-REFERENCE TO RELATED APPLICATIONS

This application claims the benefit of copending provisional application serial No. 60/365,285 filed on Mar. 19, 2002.

### STATEMENT REGARDING FEDERALLY SPONSORED RESEARCH OR DEVELOPMENT

Not applicable.

### BACKGROUND OF THE INVENTION

The present invention generally relates to emollient gels used for cosmetic and pharmaceutical applications. Emollients used for skin care applications typically fall into one of three categories: low-spreading emollients, medium-spreading emollients and high-spreading emollients. While these types of emollients are useful for certain applications relating to the treatment of skin, they each have their drawbacks, particularly the low-spreading emollients.

For many applications such as, for example, the application of sunscreen protection onto skin, low-spreading emollients are oftentimes used. The problem, however, is the attendant oily and sticky feeling imparted onto the skin by these conventionally used low-spreading emollients. Consumers, in general, prefer a dry, light feeling on their skin rather than a sticky, heavy sensation.

There is thus a need for an emollient gel that combines the desirable physical profile of being both dry feeling and low-spreading.

### BRIEF SUMMARY OF THE INVENTION

The present invention is directed to a low-spreading, dry emollient gel containing:

(a) an emollient component;

(b) a solid particulate component; and

(c) a carbohydrate derivative.

The present invention is also directed to a process for treating skin involving contacting the skin with a low-spreading, dry emollient gel containing:

(a) an emollient component;

(b) a solid particulate component; and

(c) a carbohydrate derivative.

### BRIEF DESCRIPTION OF THE SEVERAL VIEWS OF THE DRAWINGS

Not applicable.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

The present invention is directed to an emollient gel composition which may be used as a carrier for cosmetic and/or pharmaceutical active ingredients. The emollient gel composition is capable of providing a low-spreading profile while imparting a dry, elegant feeling onto skin contacted therewith. The ingredients necessary for making the emollient gel composition include an emollient component, a silica component and a carbohydrate derivative.

Suitable emollient components which may be employed in the present invention include both natural and synthetic emollients commonly known in the art. The emollient components should possess a spreading profile, on human skin, of at least about 500 mm²/10 min. Examples of these types of emollients include, but are not limited to, fatty acid esters, fatty acid ethers, glycerols, glycols, fatty carbonates and mixtures thereof. The emollient component will typically be present in the composition in an amount of from about 50 to about 90% by weight, preferably from about 60 to about 80% by weight, and most preferably about 70% by weight, all weights being based on the total weight of the emollient composition.

While any conventional solid particulate component typically employed in cosmetic and/or pharmaceutical products as a thickening agent may be used, silica, whether fumed or in the form of silicone dioxide, is preferred. The silica component will typically be present in the composition in an amount of from about 2 to about 15% by weight, preferably from about 4 to about 12% by weight, and most preferably about 10% by weight, all weights being based on the total weight of the composition.

The final necessary ingredient for the emollient gel composition of the present invention is a carbohydrate derivative, preferably, aluminum starch octenyl succinate. Its derivation is well known in the art. The aluminum starch octenyl succinate will typically be present in the composition in an amount of from about 1 to about 40% by weight, preferably from about 10 to about 20% by weight, and most preferably about 15% by weight, all weights being based on the total weight of the composition.

The above-disclosed composition may be easily formulated by simply mixing, at room temperature, all of the necessary ingredients in order to form a stable emollient gel. Any type of conventional mixing apparatus commonly used in the art may be employed.

Additional ingredients may be added to the above-disclosed emollient gel in order to further enhance its handling properties. Examples thereof include, but are not limited to, vitamin E, sunscreen, fragrance, and the like. Similarly, various types of active ingredients used for treating skin disorders may also be added, since the emollient gel of the present invention can act as a carrier material for active ingredients such as those used for pharmaceutical purposes.

The present invention will be better understood from the examples which follow, all of which are intended for illustrative purposes only, and are not meant to unduly limit the scope of the invention in any way.

### EXAMPLES

Four emollient gels were prepared in accordance with the present invention, the precise formulation of each being found in Table 1, below.

| | | Aluminum starch | |
|---|---|---|---|
| | Emollient | octenyl succinate | fumed silica |
| Ex. 1 | CETIOL ® OE - 70% | 20% | 10% |
| Ex. 2 | CETIOL ® CC - 70% | 20% | 10% |
| Ex. 3 | MYRITOL ® 331 - 70% | 20% | 10% |
| Ex. 4 | CETIOL ® B - 70% | 20% | 10% |

CETIOL ® OE is a dicaprylyl ether commercially available from Cognis Corporation.
CETIOL ® CC is a dicaprylyl carbonate commercially available from Cognis Corporation.
MYRITOL ® 331 is a cocoglyceride commercially available from Cognis Corporation.
CETIOL ® B is a dibutyl adipate commercially available from Cognis Corporation.

The ingredients of each of the examples were mixed in a blender, at room temperature, for approximately 10 to 20 minutes. All four samples were then applied onto human skin in order to evaluate their feel and spreadability. They were all found to possess minimal greasiness and low spreadability.

What is claimed is:

1. A dry feeling, low-spreading emollient gel composition comprising:
(a) from about 50 to about 90% by weight of an emollient component;
(b) from about 1 to about 40% by weight of a solid particulate component; and
(c) from about 2 to about 15% by weight of a thickening agent, all weights being based on the total weight of the composition.

2. The composition of claim 1 wherein the emollient is present in the composition in an amount of about 70% by weight, based on the weight of the composition.

3. The composition of claim 1 wherein the emollient component is selected from the group consisting of a fatty acid ester, a fatty acid ether, a glycerol, a glycol, a fatty carbonate, and mixtures thereof.

4. The composition of claim 1 wherein the carbohydrate derivative is aluminum starch octenyl succinate.

5. The composition of claim 1 wherein the carbohydrate derivative is present in the composition in an amount of about 15% by weight, based on the weight of the composition.

6. The composition of claim 1 wherein the solid particulate component is silica.

7. The composition of claim 1 wherein the solid particulate component is fumed silica.

8. The composition of claim 1 wherein the solid particulate component is present in the composition in an amount of about 10% by weight, based on the weight of the composition.

9. A process for treating skin comprising contacting the skin with an emollient gel composition containing:
(a) from about 50 to about 90% by weight of an emollient component;
(b) from about 1 to about 40% by weight of a carbohydrate derivative; and
(c) from about 2 to about 15% by weight of a solid particulate component, all weights being based on the total weight of the composition.

10. The process of claim 9 wherein the emollient is present in the composition in an amount of about 70% by weight, based on the weight of the composition.

11. The process of claim 9 wherein the emollient component is selected from the group consisting of a fatty acid ester, a fatty acid ether, a glycerol, a glycol, a fatty carbonate, and mixtures thereof.

12. The process of claim 9 wherein the carbohydrate derivative is aluminum starch octenyl succinate.

13. The process of claim 9 wherein the carbohydrate derivative is present in the composition in an amount of about 15% by weight, based on the weight of the composition.

14. The process of claim 9 wherein the solid particulate component is silica.

15. The process of claim 9 wherein the solid particulate component is fumed silica.

16. The process of claim 9 wherein the thickening agent is present in the composition in an amount of about 10% by weight, based on the weight of the composition.

17. A cosmetic or pharmaceutical formulation comprising the emollient gel composition of claim 1.
